(19) United States
(12) Patent Application Publication
(10) Pub. No.: US 2026/0071949 A1
(43) Pub. Date: Mar. 12, 2026

(54) VISUALIZING PRESENCE OF IONIC ELECTRICALLY CHARGED PARTICLES INCLUDING VIRUS PARTICLES AND BACTERIA PARTICLES ON A SURFACE

(71) Applicants: Liliana MARR, Manhattan Beach, CA (US); Emerson MARR, Manhattan Beach, CA (US); Harry Bourne MARR, JR., Manhattan Beach, CA (US)

(72) Inventors: Liliana MARR, Manhattan Beach, CA (US); Emerson MARR, Manhattan Beach, CA (US); Harry Bourne MARR, JR., Manhattan Beach, CA (US)

(21) Appl. No.: 18/829,116

(22) Filed: Sep. 9, 2024

(51) Int. Cl. G01N 15/1434; G01N 15/01; G01N 15/1429; G01N 33/569

(52) U.S. Cl. CPC G01N 15/1434; G01N 15/01; G01N 15/1429; G01N 33/56911; G01N 33/56983; G01N 2015/1445

(57) ABSTRACT

Visualizing the presence of virus and/or bacteria particles on a surface is described. This includes transmitting one or more millimeter wave and/or terahertz output signals toward the surface. The one or more output signals may trigger a resonant frequency of an ionic electrically charged particle with an electro-chemical valence shell on the surface, such as a virus particle or a bacteria particle. Or the one or more output signals may be absorbed by such a particle. A location of the particle is determined based on a triggered frequency dependent electromagnetic response received from the particle, or a relative lack of received energy at one or more frequencies of the output signals absorbed by the particle. A visual representation of the particle on the surface is generated based on the location.

### BACKGROUND

### 1. Field

The present disclosure relates to visualizing presence of ionic electrically charged particles including virus particles and bacteria particles on a surface.

### 2. Description of the Related Art

Detection of virus and bacteria particles has traditionally occurred using a swab chemical test in which a swab is used to collect a sample of genetic material from a human, or a sample from a surface area of interest. The material collected on the swab is tested, which requires waiting to receive results indicating whether virus and/or bacteria particles are present. Other detection techniques including light microscopy, immunofluorescence detection, super resolution microscopy, and/or electron microscopy are less frequently used for detecting the presence of a virus and/or bacteria. However, none of these techniques produce a real-time visual output that may be used to determine the presence of virus and/or bacterial particles on a surface.

### SUMMARY

The following is a non-exhaustive listing of some aspects of the present techniques. These and other aspects are described in the following disclosure.

Visualizing the presence of virus and/or bacteria particles on a surface is described. This includes transmitting one or more millimeter wave and/or terahertz output signals toward the surface. The one or more output signals may trigger a resonant frequency of an ionic electrically charged particle with an electro-chemical valence shell on the surface, such as a virus particle or a bacteria particle. Or the one or more output signals may be absorbed by such a particle. A location of the particle is determined based on a triggered frequency dependent electromagnetic response received from the particle, or a relative lack of received energy at one or more frequencies of the output signals absorbed by the particle. A visual representation of the particle on the surface is generated based on the location.

Some aspects include a method for visualizing the presence of virus and/or bacteria particles on a surface. The method comprises transmitting one or more millimeter wave and/or terahertz output signals toward a surface. The one or more output signals are configured to trigger a resonant frequency of an ionic electrically charged particle with an electro-chemical valence shell on the surface. The ionic electrically charged particle with electro-chemical valence shell may comprise a virus particle or a bacteria particle. The method comprises determining a location of the particle based on a triggered frequency dependent electromagnetic response received from the particle and generating a visual representation of the particle on the surface based on the location.

In some embodiments, the method comprises determining the locations and generating visual representations of a plurality of virus and/or bacteria particles on the surface based on triggered frequency dependent electromagnetic responses received from the plurality of virus and/or bacteria particles.

In some embodiments, the method comprises transmitting of the one or more output signals toward a localized area of the surface suspected of containing the virus particles and/or the bacteria particles.

In some embodiments, the electromagnetic response is received from virus particles and/or the bacteria particles, but not other particles, such that responsive to no virus particles and/or bacteria particles being present in the localized area, no electromagnetic response is received.

In some embodiments, the one or more millimeter wave and/or terahertz output signals comprise millimeter wave radar output signals.

In some embodiments, the one or more millimeter wave and/or terahertz output signals comprise frequencies in a range of from about 1 Hz to about 3,000,000 GHz.

In some embodiments, the method comprises processing the electromagnetic response to determine a peak frequency of the electromagnetic response from the particle, and determining a type of the particle based on the peak frequency.

In some embodiments, the type of the particle comprises a type of virus, a type of bacteria, or a type of individual ionic particle.

In some embodiments, the location is a three-dimensional (3D) location of the particle.

In some embodiments, the transmitting is performed by a nanometer scale complementary metal-oxide-semiconductor (CMOS) processor generating control signals to cause a phased array of antenna elements to generate the one or more output signals.

In some embodiments, the processor is configured to direct a digitally synthesized waveform through a digital to analog converter, and into the phased array of antenna elements for transmission. In some embodiments, each of the phased array of antenna elements includes a phase shifter, a heterodyne series of up-converters where a high frequency local oscillator running at sub terahertz frequencies goes into a silicon power amplifier and into a silicon antenna element.

In some embodiments, the phased array is small enough to be included on an augmented reality device or worn on clothing.

In some embodiments, the processor tunes the local oscillator, and/or switches in multiple local oscillators to sweep a known set of frequencies at which ionic electrically charged particles with electro-chemical valence shells resonate.

In some embodiments, determining the location of the particle comprises: receiving the electromagnetic response from the particle with a millimeter wave and/or terahertz receiver, and processing the electromagnetic response with the processor to determine whether a strength of the electromagnetic response breaches a threshold. Responsive to a threshold breach, a radar ranging technique is used to determine a range of the particle from the phased array, the phase shifters are tuned to generate a beam usable to map dimensional coordinates on the surface from where the electromagnetic response was received, and beam and range gates are swept in three dimensional space to map a point cloud of electromagnetic responses.

In some embodiments, the method comprises tagging, with the processor, the point cloud with a peak frequency associated with the point cloud, and correlating the peak frequency to a likely virus or bacteria type for the particle.

In some embodiments, the radar ranging technique comprises phase shift keying, range gates, and/or pulse doppler techniques.

In some embodiments, generating the visual representation of the particle on the surface based on the location comprises rendering the point cloud in three dimensional space with the processor, and displaying the rendered point cloud.

In some embodiments, displaying the rendered point cloud comprises projecting the rendered point cloud onto the surface with an augmented reality projector.

In some embodiments, the rendered point cloud is displayed on the surface using augmented reality headsets, glasses or goggles.

In some embodiments, generating the visual representation of the particle on the surface is performed in real-time or near real-time while a user views the surface.

Other aspects include another method for visualizing presence of virus and/or bacteria particles on a surface. The method comprises transmitting one or more millimeter wave and/or terahertz output signals toward a surface. The one or more output signals are configured to be absorbed by an ionic electrically charged particle with an electro-chemical valence shell on the surface. The ionic electrically charged particle with electro-chemical valence shell may comprise a virus particle or a bacteria particle. The method comprises determining the location of the particle based on a frequency dependent electromagnetic response of the output signals reflected off of the surface. The frequency dependent electromagnetic response comprises a relative lack of energy at one or more frequencies of the output signals absorbed by the particle. The method comprises generating a visual representation of the particle on the surface based on the location.

In some embodiments, the location of the particle is determined using differential absorption spectroscopy.

In some embodiments, the method comprises processing the electromagnetic response to determine an absorbed frequency indicated by the electromagnetic response, and determining a type of the particle based on the absorbed frequency.

In some embodiments, the location is a three-dimensional (3D) location of the particle, and generating the visual representation of the particle on the surface comprises projecting a rendered point cloud that represents the particle onto the surface with an augmented reality projector; or displaying an image of the particle on the surface using augmented reality headsets, glasses or goggles.

Additional aspects include a system, including a transmitter, a receiver, one or more processors, memory, and/or other components configured to perform one or more operations of the method(s) described above. In some embodiments, the memory stores instructions that when executed by the one or more processors cause the one or more processors and/or other components of the system to effectuate operations of the above-mentioned method(s).

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1 provides a schematic illustration of an embodiment of a present system configured for visualizing presence of virus and/or bacteria particles on a surface.

FIG. 2 is a schematic illustration of components of an embodiment of the present system.

FIG. 3 is a diagram that illustrates an exemplary computing environment in accordance with embodiments of the present system.

FIG. 4 illustrates a graphical illustration of resonance transmissibility.

FIG. 5 illustrates a point cloud rendered in three dimensional (3D) visualization software.

FIG. 6A illustrates a first example of what may be visually presented via augmented reality glasses or goggles using the present system.

FIG. 6B illustrates a second example of what may be visually presented, but using a smartphone.

FIG. 7 is a flow chart that illustrates a method for visualizing presence of virus and/or bacteria particles on a surface by triggering a resonant frequency of an ionic electrically charged particle with an electro-chemical valence shell on a surface.

FIG. 8 is a flow chart that illustrates a method for visualizing presence of virus and/or bacteria particles by determining whether energy is absorbed by an ionic electrically charged particle with an electro-chemical valence shell on the surface.

While the invention is susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF CERTAIN EMBODIMENTS

To mitigate the problems described herein, the inventors had to both invent solutions and, in some cases just as importantly, recognize problems overlooked (or not yet foreseen) by others in the field of detection of virus and bacteria particles. The inventors wish to emphasize the difficulty of recognizing those problems that are nascent and will become much more apparent in the future should trends in industry continue as the inventor expects. Further, because multiple problems are addressed, it should be understood that some embodiments are problem-specific, and not all embodiments address every problem with traditional systems described herein or provide every benefit described herein. That said, improvements that solve various permutations of these problems are described below.

For example, detection of the presence of virus and bacteria particles has traditionally occurred using a swab chemical test in which a swab is used to collect a sample from a human or a surface area (which can only cover a small portion of that area). The tester has to physically touch the swab and be in close proximity to the virus and/or bacteria particles, time for sample prep and analysis is required, and results indicating presence of a virus or bacteria are received only after this time-consuming process is complete. Detection techniques such as light microscopy and immunofluorescence also require significant sample preparation time, and are unable to provide a display without microscopic equipment. Other detection techniques including electron microscopy and crystallography can display the exterior structure of virus and/or bacteria particles, which is important because understanding the structures and how they interact with human cells and antibodies informs scientist on what type of vaccine and drug designs should be created to fight off the virus particles. However, using swab chemical tests, light microscopy, immunofluorescence, electron microscopy, crystallography, and other detection techniques are time intensive, require expensive microscopic equipment and cannot be visualized in real-time, which limits early detection of virus and/or bacteria particles.

Especially with the global pandemic emerging in 2020, an increased focus on understanding the sanitization level of the environment has become increasingly important. However, with virus and bacteria, there is often no easy, fast, and practical way to detect the presence of such particles (e.g., as described above). A person must instead use preventative measures such as a mask, sanitization cleanser, or vacating a premises out of precaution, sometimes just based on suspicion of viral and/or bacterial particles being present.

Advantageously, the present systems and method utilize millimeter wave (mmW) technology and terahertz (THz) technology to facilitate real-time visualization of viral, bacterial, and/or other particles. This technology can trigger the resonant frequency of ionic particles, or particles with electro-chemical valence shells such as is the case with virus and bacteria. Integration with augmented reality, where detections using millimeter wave technology, such as millimeter wave radar, at the microscopic level can be merged with augmented reality technology, such as a headset, glasses, goggles, and/or other projection or display devices to create systems and methods can enable a user to visualize whether virus and/or bacteria particles are present on a surface (e.g., no expensive microscope equipment required). Because of the specificity with which the resonant response is triggered for a given virus, and since the response is frequency dependent, with the use of signal processing, artificial intelligence, and/or other classification algorithms, the type of virus, bacteria, or individual ionic particle can be determined.

Enabling real-time instant detection based on a resonant frequency, and visualizing a concentration of virus and/or bacterial particles (Covid 19 particles as one example) using augmented reality devices and point clouds (as described below) allows a person to take preventative measures only as needed, as one example (e.g., cleaning a surface, putting on a mask, using sanitization cleanser, vacating the premises out of precaution—all of which prevent hospitalization). This technology could be employed to detect particles on surfaces of medical equipment, on floors, on windows, on computers, on tables, on desks, on chairs, on mobile devices such as mobile phones, etc., in hospitals, businesses, homes, churches, community centers, schools, elderly homes, outdoors, etc. to assess for the presence of COVID-19 and/or other virus and/or bacteria particles, for example.

Much work has been done on understanding the resonant frequencies of virus particles, which are known to be in the microwave and mmW bands (1-2000 GHz). Some work has been done using electromagnetic waves to potentially break the cuspid shell of the virus itself, if a strong enough electric potential is applied, to “cure” the virus. Some work has been done determining precise frequencies that have a much stronger signal response than other frequencies, which correspond to different types of virus and/or bacteria particles, including Covid 19 which has a peak frequency. However, all of this work is focused on treating or killing a particular virus and/or bacteria, such as by irradiating the virus and/or bacteria particle to kill it. Again, none of this work has provided a real-time visualization technique for virus and/or bacteria particles on surfaces.

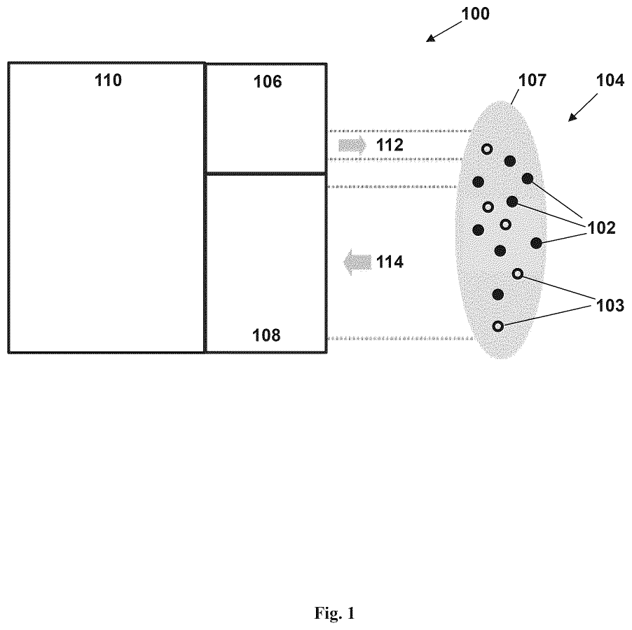

FIG. 1 provides a schematic illustration of system 100 for visualizing presence of virus and/or bacteria particles 102 (as compared to other non-virus or non-bacterial particles 103) on a surface 104. System 100 comprises a transmitter 106, a receiver 108, a processor 110, and/or other components. System 100 is configured to determine the location(s) of particles 102, and generate visual representations of the particles 102 on surface 104 based on the locations and/or other information. This may include rendering a point cloud in three dimensional space to represent different particles 102, and displaying the rendered point cloud or some other visual representation. System 100 may be included in and/or otherwise associated with an augmented reality projector configured to project the rendered point cloud onto surface 104, augmented reality glasses or goggles configured to display the rendered point on surface 104, a user's smartphone which can display a visual representation of particles 102, and/or other devices. System 100 is configured such that visual representation of the particle(s) 102 on surface 104 is performed in real-time or near real-time while a user of system 100 views surface 104. Each of the components of system 100 is described in turn below.

Transmitter 106 is configured to transmit one or more millimeter wave and/or terahertz output signals 112 toward surface 104. The one or more millimeter wave and/or terahertz output signals may comprise millimeter wave radar output signals, for example. The output signals may have frequencies in a range of from about 1 Hz to about 3,000,000 GHz. Surface 104 may be and/or include a surface of a smartphone, a table, a computer, a desk, a door handle, etc., for example (there are many more possible examples of surfaces 104). Transmitter 106 is configured to transmit one or more output signals toward one or more localized areas 107 of surface 104 suspected of containing the virus particles and/or the bacteria particles 102. The one or more output signals 112 may be configured to trigger a resonant frequency of an ionic electrically charged particle with an electro-chemical valence shell on surface 104. Virus and/or a bacteria particle(s) 102 are examples of ionic electrically charged particles with electro-chemical valence shells. A resonant frequency may include mechanical resonance, orbital resonance, acoustic resonance, electromagnetic resonance, nuclear magnetic resonance, electron spin resonance and resonance of quantum wave functions, for example.

Receiver 108 is configured to receive a triggered frequency dependent electromagnetic response 114 from the particle(s) 102. Receiver 108 is a millimeter wave and/or terahertz receiver. The electromagnetic response may be received from virus particles and/or the bacteria particles 102, but not other particles, such that responsive to no virus particles and/or bacteria particles being present in the localized area, no electromagnetic response is received. Receiver 108 may generate an electronic signal (e.g., for processor 110) comprising information indicative of the received electromagnetic response(s) from particle(s) 102. Note that transmitter 106 and receiver 108 may be formed from the same or similar overlapping components, as described below.

Processor 110 may be a nanometer scale complementary metal-oxide-semiconductor (CMOS) and/or other processors. Processor 110 is configured to determine a location of the (e.g. virus and/or bacteria particle(s)) 102 based on the triggered frequency dependent electromagnetic response 114 and/or other information. In some embodiments, processor 110 is configured to process electromagnetic response 114 to determine a peak frequency of electromagnetic response 114 from each of the particles 102, and determine a type of the particle 102 based on the peak frequency and/or other information. The type of the particle(s) 102 comprises a type of virus, a type of bacteria, or a type of individual ionic particle, for example. Processor 110 may also or instead determine a concentration of particles 102 and/or other information based on electromagnetic response 114.

Processor 110 is configured to generate a visual representation (described below) of the particle(s) 102 on surface 104 based on the determined location(s) and/or other information. The location is a three-dimensional (3D) location of the particle 102, for example. Processor 110 may be configured to determine locations and generate visual representations of a plurality of virus and/or bacteria particles 102 on surface 104 based on triggered frequency dependent electromagnetic responses received from the plurality of virus and/or bacteria particles 102 and/or other information.

In some embodiments, the one or more output signals 112 are configured to be absorbed by the virus and/or bacteria particles 102 (e.g., ionic electrically charged particles with electro-chemical valence shells on the surface). Receiver 108 is configured to receive the frequency dependent electromagnetic response 114 from surface 104, but the frequency dependent electromagnetic response 114 may instead comprise a relative lack of energy at one or more frequencies of the output signals absorbed by the particle(s) 102. Processor 110 is again configured to determine the location(s) of the particle(s) 102 based on the frequency dependent electromagnetic response 114, but now the location is determined based on the relative lack of energy at a certain frequency (or frequencies). In these embodiments, processor 110 may use differential absorption spectroscopy and/or other techniques to determine a location. Processor 110 may again be configured to determine locations and generate visual representations of a plurality of virus and/or bacteria particles 102 on surface 104, but based on absorbed frequency dependent electromagnetic responses 114 received from the plurality of virus and/or bacteria particles 102.

Processor 110 (which may comprise one or more processors 110) is configured to provide information processing capabilities in system 100. As such, processor(s) 110 may include one or more of a digital processor, an analog processor, a digital circuit designed to process information, an analog circuit designed to process information, a state machine, and/or other mechanisms for electronically processing information. In some embodiments, a processor 110 may be included in and/or otherwise operatively coupled with transmitter 106, receiver 108, a computing device (e.g., as described below), and/or other components of system 100. Although one or more processors 110 are shown in FIG. 1 as a single entity, this is for illustrative purposes only. In some embodiments, processor(s) 110 may include a plurality of processing units. These processing units may be physically located within the same device (e.g., a single device together with transmitter 106 and receiver 108), a computing device (e.g., an augmented reality display device, as described below), or processor(s) 110 may represent processing functionality of a plurality of devices operating in coordination (e.g., a processor located within transmitter 106, a second processor located within receiver 108, and a third processor located in an augmented reality display device—which all may be together considered one system 100 whether formed as once single component or separate components). Processor(s) 110 may be configured to execute one or more computer program components. Processor(s) 110 may be configured to execute the computer program component by software; hardware; firmware; some combination of software, hardware, and/or firmware; and/or other mechanisms for configuring processing capabilities on processor(s) 110.

One or more processors 110 are configured to control transmitter 106, receiver 108, and/or other components of system 100. Control may comprise electronic communication of one or more commands to transmitter 106 and/or receiver 108, and/or other control operations. For example, processor(s) 110 are configured to cause transmitter 106 to emit output signals 112 toward surface 104. Processor(s) 110 may cause transmitter 106 to adjust a wavelength, frequency, and/or other characteristics of output signals 112. In some embodiments, processor(s) 110 may cause transmitter 106 to sweep a series of wavelengths and/or frequencies, for example. Processor(s) 110 can cause transmitter 106 to sweep the frequency of the mmW/THz output signals across a library of known virus and/or bacteria frequency responses and tune receiver 108 to detect any of these frequency responses. The “beam” can be swept over a large area with instantaneous results and has a range of several meters up to several hundred meters so that a physical surface does not need to be touched. This may be based on a virus and/or bacteria particle 102 detection, and/or other information. Processor(s) 110 may be configured such that controlling transmitter 106 comprises causing transmitter 106 to generate and/or modulate (e.g., as described herein), an amount, a timing, a wavelength, a frequency, and/or intensity of output signals 112 at spatially localized areas of surface 104. In some embodiments, one or more processors 110 are configured such that detecting the viral and/or bacterial particles 102 occurs in real time as system moves proximate to surface 104 (as described herein).

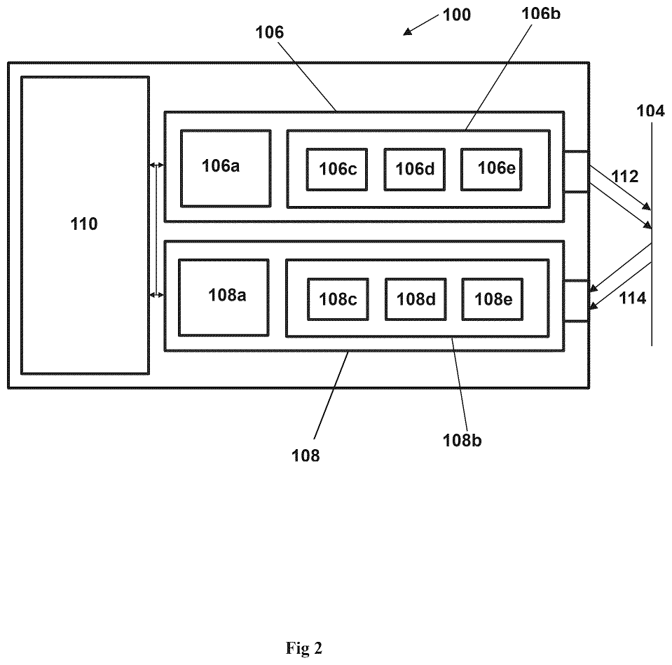

FIG. 2 provides a more detailed view of system 100. System 100 comprises a transmitter 106, a receiver 108, a processor 110 (or a plurality of processors 110), and/or other components as described above in FIG. 1. The detailed components of transmitter 106 and receiver 108 are described in turn below.

Transmitter 106 includes a digital to analog converter 106a and a phased array 106b of antenna elements. Many hundreds of elements can be built into a phased array 106b. Each of the phased arrays 106b of antenna elements includes a phase shifter 106c, a heterodyne series of up-converters 106d where a high frequency local oscillator running at sub terahertz frequencies goes into a silicon power amplifier and into a silicon antenna element, and/or other components. Processor 110 is configured to direct a digitally synthesized waveform through digital to analog converter 106a, and into the phased array 106b of antenna elements for transmission. Digital to analog converter 106a is configured to receive a digitally synthesized waveform from processor 110. Digital to analog converter 106a maps the digitally synthesized waveform, which may comprise a finite number of digital input bits in binary number representation for example, to a corresponding set of continuous analog output voltages or current levels. For example, a resistor may be connected to each bit position of the digitally synthesized wave form input. Each resistor may be connected to a switch which is controlled by each bit position of the digitally synthesized wave form input. Switches selectively connect the resistors to a reference voltage which is summed up to generate the analog output voltage. Once the voltage is summed up to generate an output voltage, The analog output voltage may go through a low-pass filter to ensure the output voltage is a continuous waveform.

A phase shifter 106c may receive an analog output signal and control a position of the analog output signal at a particular timeframe for directing the one or more millimeter wave and/or terahertz output signals 112 (e.g., “beams”) from transmitter 106 a certain direction, for example. The analog output signal may be transmitted from processor 110 to an up-converter 106d. Up-converters 106d may be configured to receive the analog output signal from phase shifters 106c. Up-converters 106d may include a local oscillator (not shown in figure) configured to generate a high-frequency analog output signal and a mixer (not shown in figure) that combines the analog output signal and the high frequency analog output signal generated by local oscillator to produce an analog output signal at two frequencies. Up-converters 106d may include a bandpass filter (not shown in figure) configured to filter the two frequencies in order for the target frequency of the two frequencies maybe transmitted and to attenuate the undesired frequency of the two frequencies. An amplifier (not shown in figure) of an up-converter 106d may be configured to receive the target frequency and provide a power gain to the analog output signal. A silicon antenna 106e may be configured to receive the analog output signal from the amplifier of up-converter 106d and transmit the analog output signal (the one or more output signals 112) to surface 104. As described above, processor 110 can tune the local oscillator, and/or switches in multiple local oscillators to sweep a known set of frequencies at which ionic electrically charged particles with electro-chemical valence shells resonate.

As described above, receiver 108 is configured to receive electromagnetic response 114, with the same or similar components described above that make up transmitter 106, just in reverse order (e.g., antenna to digital signal conversion for processor 110). Receiver 108 may include an analog to digital converter 108a (e.g., the same component as digital to analog converter 106a, just functioning in reverse), a phased array 108b, and/or other components. Phased array 108b may include phase shifter(s) 108c, down-converter(s) 108d which include local oscillator(s), power amplifier(s), silicon antenna(s) 108e, and/or other components. In some embodiments, receiver 108 may comprise substantially the same or similar components as the components in transmitter 106 (but which function in a receiving capacity, to provide received information to processor 110).

Processor 110 is configured to process the electromagnetic response 114 from the virus or bacteria particles 102 (as examples of ionic electrically charged particles with electro-chemical valence shells on their surfaces). In some embodiments, processor 110 may determine whether a strength of the electromagnetic response 114 breaches a threshold. Responsive to a threshold breach, processor 110 may be configured to use a radar ranging technique to determine a range of a particle 102 (FIG. 1) from phased array 106b and/or 108b, tune the phase shifters 106c to generate a millimeter wave/THz beam (that can be as narrow as a laser, for example) usable to map dimensional coordinates on surface 104 from where the electromagnetic response 114 was received, cause sweeping of beam and range gates in three dimensional space to map a point cloud of electromagnetic responses, and/or perform other actions. These techniques can be used to understand the various dimensional coordinates of where the electromagnetic response(s) 114 came from, for example, on the surface. Phrased another way, this beam may be used to map dimensional coordinates on surface 104 from where electromagnetic responses 114 ware received. The radar ranging technique may comprise phase shift keying, range gates, pulse doppler techniques, and/or other techniques. A point cloud can indicate the exact 3D location of a particle (and a plurality of point clouds can indicate the locations of a plurality of particles). The beam and range gates may be swept in 3D space to map all point cloud detections of electromagnetic response(s) 114, for example. Each point cloud can be tagged by processor 110 with a peak frequency. In some embodiments, processor 110 is configured to map the frequency response (e.g., a peak frequency) to a likely virus type.

For example, processor 110 may control transmitter 106 to sweep the frequency of the mmW/THz output signals 112 across a library of known virus and/or bacteria particle electromagnetic responses 114, and tune receiver 108 to detect any corresponding electromagnetic responses 114. Mapping may include using artificial intelligence techniques, maximum likelihood ratio techniques and/or other techniques, for example. As described above, the radar beam may be swept over a large area with instantaneous results and may have a range of several meters up to several hundred meters so that physical surface does not need to be touched, for example.

Similarly, in some embodiments, processor 110 is configured to determine the location of a particle or particles based on absorption of output signals 112 indicated in the electromagnetic response 114 (e.g., a lack of signal strength which does not breach the threshold described above). Processor 110 may determine the location of a particle, sweep frequencies, generate a point cloud, tag a point cloud (e.g., with an absorbed frequency), and/or perform other operations based on a relative lack of energy at one or more frequencies of the output signals 112 absorbed by the particle(s). For example, the location of a particle may be determined using differential absorption spectroscopy and/or other techniques by processor 110. Processor 110 may be configured to process an electromagnetic response 114 to determine an absorbed frequency indicated by the electromagnetic response 114, and determine a type of the particle based on the absorbed frequency and/or other information.

System 100, including transmitter 106, receiver 108, processor 110, some and/or all of the various subcomponents described above, and/or other components may small enough (e.g., an inch square or less in size) to be included in an augmented reality device, worn on clothing, included in a user's smartphone and/or other computing device, and/or incorporated into or onto other object, for example. Incorporating system 100 into other objects like these can facilitate the real-time convenience described herein. For example, if a user can simply direct their smartphone, or augmented reality glasses, or similar device, toward surface 104 and visually observe (e.g., as described below) virus or bacteria particles, the user can immediately take action to clean or avoid surface 104 (as two potential example responses of many).

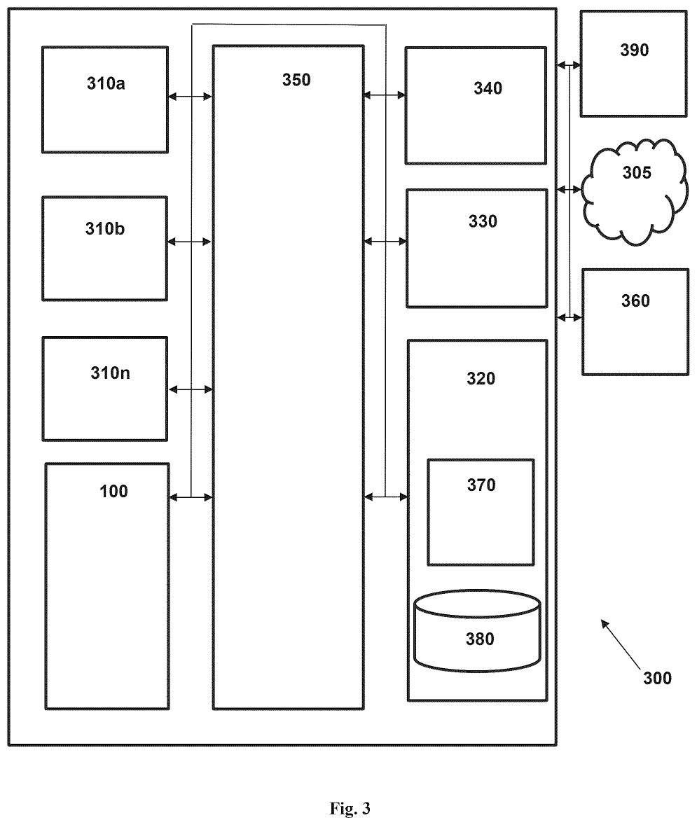

FIG. 3 is a diagram that illustrates an exemplary computing device 300 (e.g., an augmented reality projector, augmented reality glasses or goggles or headset, a smartphone, a laptop, a gaming system, etc.) that may include, be coupled to, and/or otherwise be associated with system 100 (FIG. 1, FIG. 2). Various portions of systems and methods described herein may be included in, or be executed on, one or more computing devices the same as or similar to computing device 300. For example, processor(s) 110 of system 100 (FIG. 1) may be and/or be included in one more computing devices the same as or similar to computing device 300. Further, processes, modules, processor components, and/or other components of system 100 described herein may be executed and/or otherwise controlled by one or more computing systems similar to and/or the same as that of computing device 300.

Computing device 300 may include one or more processors (e.g., processors 310a-310n, which may include and/or be similar to and or the same as processor(s) 110). The processors may be operatively coupled to system memory 320, an input/output I/O device interface 330, a network interface 340, an I/O interface 350, and/or other components. A processor (e.g., 310a, 310b, . . . 310n, 110 (FIG. 1)) may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing device 300. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 320). Computing device 300 may be a uni-processor system including one processor (e.g., processor 310a), or a multi-processor system including any number of suitable processors (e.g., 310a-310n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing device 300 may include a plurality of computing devices (e.g., distributed computer systems) to implement various processing functions. A processor may execute code and store data on a server and/or retrieve data from server to store and/or execute on one or more processors.

I/O device interface 330 may provide an interface for connection of one or more I/O devices 360 to computer device 300. I/O devices may include devices that receive input (e.g., from a user) or output information (e.g., to a user). I/O devices 360 may include, for example, graphical user interface presented on displays (e.g., a liquid crystal display (LCD) monitor, a touchscreen, the display of augmented reality glasses or goggles or headset, etc.), pointing devices (e.g., a computer mouse or trackball), keyboards, keypads, touchpads, scanning devices, voice recognition devices, gesture recognition devices, printers, audio speakers, microphones, cameras, or the like. I/O devices 360 may be connected to computing device 300 through a wired or wireless connection. I/O devices 360 may be connected to computing device 300 from a remote location. I/O devices 360 located on remote computer system, for example, may be connected to computing device 300 via a network and network interface 340.

Computing device may be configured to access external resources 390, which in some embodiments, include sources of information such as databases, websites, etc ; external entities participating with computing device 300 and/or system 100 (e.g., systems or networks associated with system 100), one or more servers outside of computing device and/or system 100, a network (e.g., the internet), electronic storage, equipment related to Wi-Fi ™ technology, equipment related to Bluetooth® technology, data entry devices, or other resources. In some implementations, some or all of the functionality attributed herein to external resources 390 may be provided by resources included in system 100. External resources 390 may be configured to communicate with one or more other components of computing device 300 and/or system 100 via wired and/or wireless connections, via a network (e.g., a local area network and/or the internet), via cellular technology, via Wi-Fi technology, and/or via other resources. For example, external resources 390 may include a database of known virus and/or bacteria particle electromagnetic responses 114 (FIG. 1, FIG. 2), as described above.

Network 305 may include the internet, a Wi-Fi network, Bluetooth® technology, and/or other wireless technology. In some embodiments, one or more processors 110 and/or 310a-310n, computing device 300, external resources 390, and/or other components of system 100 communicate via network 305, This may include near field communication, Bluetooth, and/or radio frequency communication, internet communication, and/other communication methods.

Network interface 340 may include a network adapter that provides for connection of computing device 300 to a network (e.g., network 305 described above). Network interface may 340 may facilitate data exchange between computing device 300 and/or system 100 and other devices connected to the network (e.g., external resources 390 which include a database of known virus and/or bacteria particle electromagnetic responses). Network interface 340 may support wired or wireless communication.

System memory 320 may be configured to store program instructions 370 (e.g., machine readable instructions) and/or other data 380. Program instructions 370 may be executable by a processor (e.g., one or more of processors 310a-310n, 110, etc.) to implement one or more embodiments of the present techniques. Instructions 370 may include modules and/or components of computer program instructions for implementing one or more techniques described herein with regard to various processing modules and/or components. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

Memory 320 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., hard-drives), or the like. Memory 320 may include a single memory device and/or a plurality of memory devices (e.g., distributed memory devices). In some cases, the entire set of instructions may be stored concurrently in memory 320, or in some cases, different parts of the instructions may be stored on the same media at different times, e.g., a copy may be created by writing program code to a first-in-first-out buffer in a network interface, where some of the instructions are pushed out of the buffer before other portions of the instructions are written to the buffer, with all of the instructions residing in memory on the buffer, just not all at the same time.

I/O interface 350 may be configured to coordinate I/O traffic between processors 310a-310n, 110, etc., memory 320, network interface 340, I/O devices 360, and/or other peripheral devices. I/O interface 350 may perform protocol, timing, or other data transformations to convert data signals from one component (e.g., memory 320) into a format suitable for use by another component (e.g., processors 310a-310n, 110, etc.). I/O interface 350 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computing device 300 or multiple computing devices 300 configured to host different portions or instances of embodiments. Multiple computing devices 300 may provide for parallel or sequential processing/execution of one or more portions of the techniques described herein.

Those skilled in the art will appreciate that computing device 300 is merely illustrative and is not intended to limit the scope of the techniques described herein. Computing device 300 may include any combination of devices or software that may perform or otherwise provide for the performance of the techniques described herein. For example, computing device 300 may include or be a combination of a cloud-computing system, a data center, a server rack, a server, a virtual server, a desktop computer, a laptop computer, a tablet computer, a server device, a client device, a smartphone, a personal digital assistant (PDA), a mobile audio or video player, a game console, a vehicle-mounted computer, augmented reality headset, glass or goggles, or the like. Computing device 300 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computing device 300 may be transmitted to computing device 300 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present invention may be practiced with other computer system configurations.

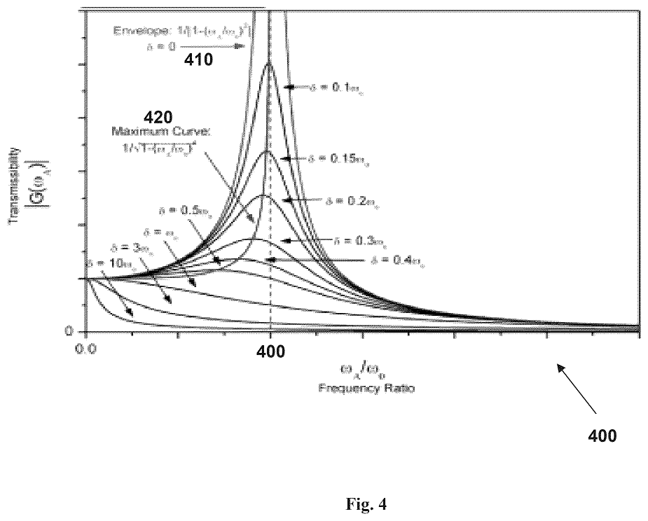

FIG. 4 provides a graphical illustration of a plot 400 of resonance transmissibility as a function of a frequency ratio. Plot 400 is a graphical representation of how to measure vibrational behaviors of particles 102 (FIG. 1), for example. Plot 400 illustrates a peak frequency 402 (ratio). This is one possible example of the frequency dependent electromagnetic response 114 described above (FIG. 1, FIG. 2). Resonance transmissibility is the ratio of an input acceleration to a response acceleration. Plot 400 illustrates an analysis of the behavior of a particle (e.g., a particle 102 shown in FIG. 1) under forced vibration (e.g., caused by one or more output signals shown in FIG. 1 and FIG. 2). Plot 400 focuses on how the particle's transmissibility varies with different values of damping coefficients (δ) and responses at various input frequencies (ω_{Δ}) relative to natural frequencies (ω_{φ}). Plot 400 depicts x and y coordinates where the x coordinate (horizontal axis) is the frequency ratio. The frequency ratio is a measure of the input frequency to the natural frequency. The input frequency comprises a number of vibrations for a given time period. Natural frequency is the frequency at which a particle 102 (FIG. 1) vibrates when there are no external forces. The y-coordinate (vertical axis) is the measure of transmissibility. Transmissibility measures how much input frequency is transmitted through a particle to output. Plot 400 depicts how, when the frequency ratio is less than 1, the transmissibility decreases while damping values increase. Plot 400 displays how when the frequency ratio is equal to 1, the particle exhibits resonance, transmissibility is at a peak, and damping is minimal. When resonance occurs, the particle's response amplitude may be amplified depending on the level of damping present. As the frequency ratio becomes greater than 1, transmissibility declines. Plot 400 depicts multiple curves corresponding to different damping coefficients. Damping is the loss of energy of an oscillating particle by dissipation. The lower the damping then the higher resonance peaks while higher damping reduces maximum transmissibility.

Plot 400 depicts curves with damping values which are used to display the effect of increasing damping on a particle's transmissibility. Curve 410 displays the maximum transmissibility when the damping is at zero. The particle is underdamped and may lead to a disastrous resonance if the input frequency matches the natural frequency. Curve 420 displays the maximum transmissibility at different damping values. As damping values increase the transmissibility decreases, which shows how damping can mitigate resonance.

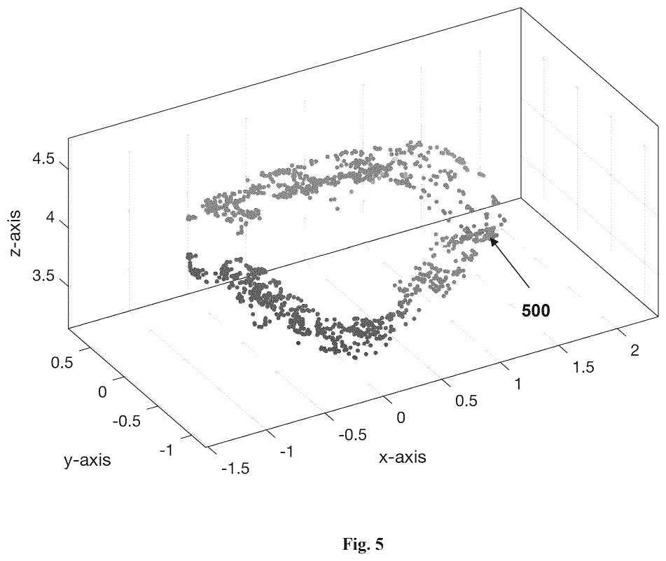

FIG. 5 illustrates a point cloud 500. Processor 110 (FIG. 1) is configured to render point cloud 500, as described above. Point cloud 500 can indicate the exact 3D location (with several x, y, and z axis coordinates) of a particle (and a plurality of point clouds can indicate the locations of a plurality of particles). Point cloud 500 may be rendered in 3D visualization software, for example, and/or using other techniques. Point cloud 500 is a collection of data points in a 3D coordinate system. Each point in the cloud represents a specific location in space defined by X, Y and Z coordinates. These coordinates may be indicated relative to the position of the surface (e.g., as a reference), and/or using other techniques. Point clouds 500 are used to represent the surface of a particle, on a surface in 3D space, for example. As described above (see FIG. 1 and/or FIG. 2), transmitter 106 transmits one or more output signals 112 to a localized area 107 of surface 104. One or more output signals 112 may be configured to trigger a resonant frequency of an ionic electrically charged particle with an electro-chemical valence shell on surface 104. Receiver 108 is configured to receive the triggered resonant frequency dependent electromagnetic response 114 from the particle(s) 102. Processor 110 configures the beam and range gates to sweep in 3D space to map all “point cloud” detections. Each point cloud is tagged with a peak frequency. Processor 110 may use software to map the frequency response 114 to the likely virus type. Processor 110 is configured to determine locations and generate visual representations of a plurality of virus and/or bacteria particles 102 on surface 104 based on triggered frequency dependent electromagnetic responses 114 received from the plurality of virus and/or bacteria particles 102. The point cloud is then rendered in 3D visualization software.

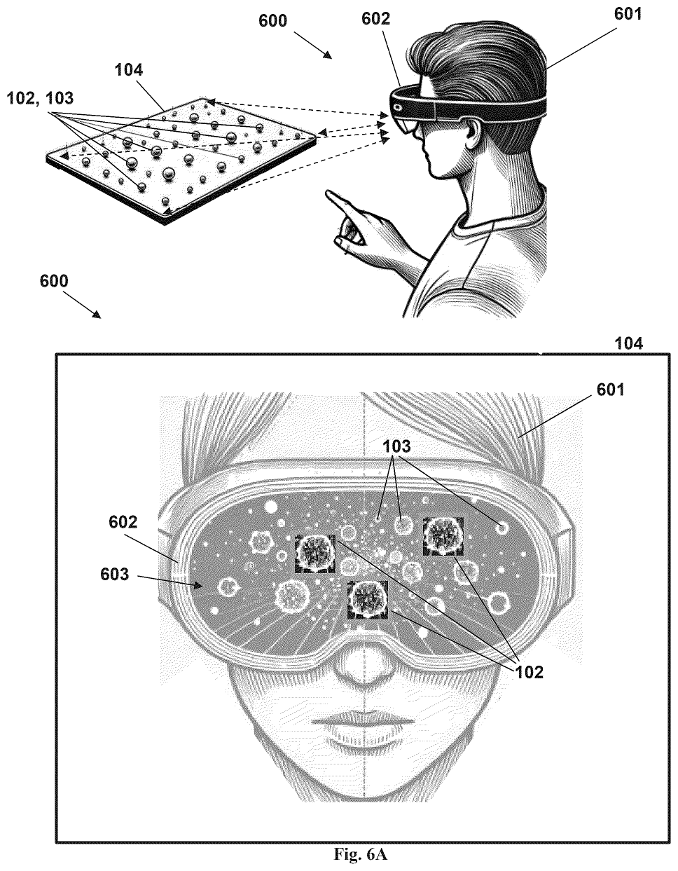

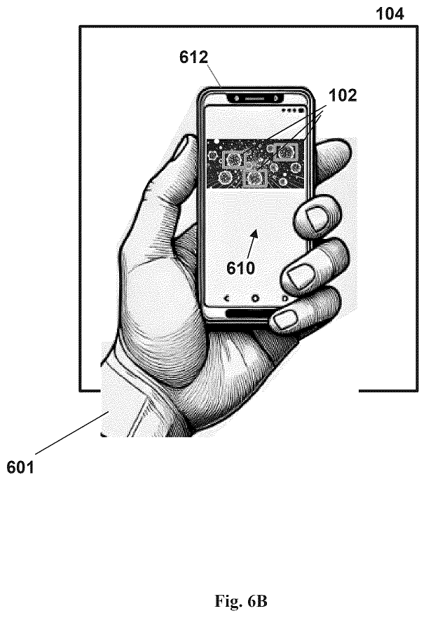

FIG. 6A and FIG. 6B provide an examples of what may be visually presented to a user 601 using the present systems and/or methods. FIG. 6A illustrates generating a visual representation of particles 102 (and particles 103) on surface 104 via augmented reality (AR) glasses or goggles 602. FIG. 6A illustrates a computing device 600 comprising augmented reality (AR) goggles 602, a display 603 within the AR goggles 602, and the virus/or bacteria particles 102 (along with non-viral or bacterial particles 103) on surface 104. FIG. 6A illustrates the particles on surface 104 in a perspective view (top), and also displayed on surface 104 (bottom—note in this view surface 104 appears transparent).

Device 600 may be similar to and/or the same as computing device 300 shown in FIG. 3 and described above (and so may be and/or include system 100 shown in FIG. 1 and FIG. 2). As described above for device 300, device 600 is not limited to augmented reality glasses or goggles 602 but may comprise an application running on a laptop or desktop computer, a smartphone, a tablet, or another computer or mobile device. For example, FIG. 6B illustrates generating a visual representation of particles 102 (and particles 103) on surface 104 via a display 610 on a smartphone 612 (user 601 is holding smartphone 612 just in front of surface 104 in this example).

In either one of these examples (referring back to the functionality described above with respect to FIG. 1 and FIG. 2) receiver 108 receives electromagnetic response 114. Processor 110 processes the electromagnetic response 114 as described above. Processor 110 determines particle location(s) and renders a point cloud (or multiple point clouds in this example) in three-dimensional space. Processor 110 is configured to generate the visual representations shown in FIG. 6A and FIG. 6B using display 603 and/or 610 of the particle(s) 102 on surface 104 based on the location(s). This displaying may also and/or instead comprise using an augmented reality projector or another device configured to project the rendered point cloud onto surface 104, for example. Processor 110 may generate the visual representation of the particle(s) 102 in real-time or near real-time while the surface 104 is viewed by user 601, for example.

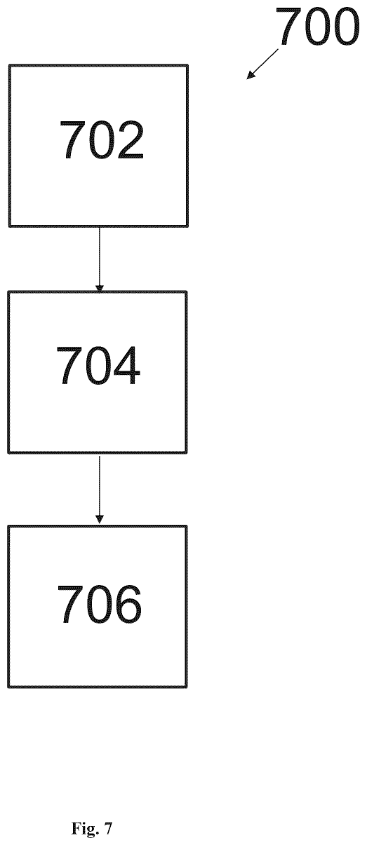

FIG. 7 illustrates a method 700 for visualizing the presence of virus and/or bacteria particles on a surface. The operations of method 700 presented below are intended to be illustrative. In some embodiments, method 700 may be accomplished with one or more additional operations not described, and/or without one or more of the operations discussed. Additionally, the order in which the operations of method 700 are illustrated in FIG. 7 and described below is not intended to be limiting.

In some embodiments, some or all of method 700 may be implemented in one or more processing devices (e.g., a digital processor, an analog processor, a digital circuit designed to process information, an analog circuit designed to process information, a state machine, and/or other mechanisms for electronically processing information). The one or more processing devices (e.g., processor(s) 110 shown in FIG. 1, processor 310a shown in FIG. 3, etc., described herein) may include one or more devices executing some or all of the operations of method 700 in response to instructions stored electronically on an electronic storage medium (e.g., system memory 320 shown in FIG. 3, etc.). The one or more processing devices may include one or more devices configured through hardware, firmware, and/or software to be specifically designed for execution of one or more of the operations of method 700.

At an operation 702, one or more millimeter wave and/or terahertz output signals are transmitted toward a surface. The one or more millimeter wave and/or terahertz output signals comprise millimeter wave radar output signals and frequencies in a range of from about 1 Hz to about 3,000,000 GHz. The one or more output signals are transmitted toward a localized area of the surface suspected of containing the virus particles and/or the bacteria particles. The transmitting is performed by a nanometer scale complementary metal-oxide-semiconductor (CMOS) processor generating control signals to cause a phased array of antenna elements to generate one or more output signals. A processor is configured to direct a digitally synthesized waveform through a digital to analog converter, and into the phased array of antenna elements for transmission. Each of the phased array of antenna elements includes a phase shifter, and a heterodyne series of up-converters where a high frequency local oscillator running at sub terahertz frequencies goes into a silicon power amplifier and into a silicon antenna element. The phased array is small enough to be included on an augmented reality device or worn on clothing. The processor tunes the local oscillator, and/or switches in multiple local oscillators to sweep a known set of frequencies at which ionic electrically charged particles with electro-chemical valence shells resonate. The one or more output signals are configured to trigger a resonant frequency of an ionic electrically charged particle with an electro-chemical valence shell on the surface. The ionic electrically charged particle with electro-chemical valence shell comprises a virus particle or a bacteria particle. In some embodiments, operation 702 is performed by or with a processor and/or a transmitter similar to and/or the same as processor 110 and/or transmitter 106 (shown in FIG. 1 and described herein).

At an operation 704, a location of the particle is determined based on a triggered frequency dependent electromagnetic response received from the particle. The location is a three-dimensional (3D) location of the particle. In some embodiments, a type of particle may be determined. The type of the particle comprises a type of virus, a type of bacteria, or a type of individual ionic particle. The electromagnetic response is received from virus particles and/or the bacteria particles, but not other particles, such that responsive to no virus particles and/or bacteria particles being present in the localized area, no electromagnetic response is received. The electromagnetic response is received from the particle with a millimeter wave and/or terahertz receiver. The electromagnetic response is processed with the processor to determine whether a strength of the electromagnetic response breaches a threshold. Responsive to a threshold breach, a radar ranging technique is used to determine a range of the particle from the phased array. The radar ranging technique comprises phase shift keying and range gates and pulse doppler techniques. The phase shifters are tuned to generate a beam usable to map dimensional coordinates on the surface from where the electromagnetic response was received. Beam and range gates are swept in three-dimensional space to map a point cloud of electromagnetic responses. The electromagnetic response is processed to determine a peak frequency of the electromagnetic response from the particle and determine a type of the particle based on the peak frequency. The processor may tag the point cloud with a peak frequency associated with the point cloud, and correlate the peak frequency to a likely virus or bacteria type for the particle In some embodiments, operation 704 is performed by or with a receiver and/or a processor similar to and/or the same as receiver 108 and/or processor 110 (shown in FIG. 1 and described herein).

At an operation 706, a visual representation of the particle is generated based on the determined location and/or other information. The visual representation may be displayed on the surface. Generating the visual representation of the particle and displaying it on the surface comprises rendering the point cloud in three-dimensional space with the processor and displaying the rendered point cloud. Displaying the rendered point cloud may comprise projecting the rendered point cloud onto the surface with an augmented reality projector as one example. The rendered point cloud may be displayed on the surface using augmented reality headsets, glasses or goggles as another example. The visual representation of the particle that is generated on the surface is performed in real-time or near real-time while a user views the surface. In some embodiments, operation 706 is performed by augmented reality headsets, glasses or goggles and/or a computing device the same as or similar to augmented reality goggles 602 and/or computing device 300 (shown in FIGS. 3 and 6A respectively and described herein).

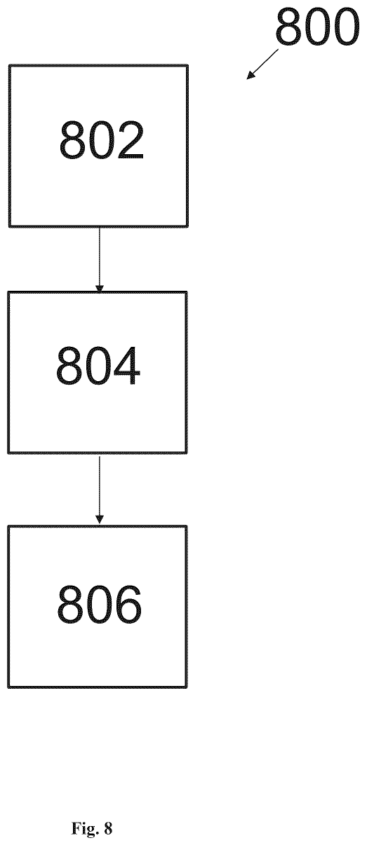

FIG. 8 illustrates another method 800 for visualizing presence of virus and/or bacteria particles on a surface. The operations of method 800 presented below are intended to be illustrative. In some embodiments, method 800 may be accomplished with one or more additional operations not described, and/or without one or more of the operations discussed. Additionally, the order in which the operations of method 800 are illustrated in FIG. 8 and described below is not intended to be limiting.

In some embodiments, some or all of method 800 may be implemented in one or more processing devices (e.g., a digital processor, an analog processor, a digital circuit designed to process information, an analog circuit designed to process information, a state machine, and/or other mechanisms for electronically processing information). The one or more processing devices (e.g., processor(s) 110, processor 310a, etc., described herein) may include one or more devices executing some or all of the operations of method 800 in response to instructions stored electronically on an electronic storage medium (e.g., system memory 320, etc.). The one or more processing devices may include one or more devices configured through hardware, firmware, and/or software to be specifically designed for execution of one or more of the operations of method 800.

At an operation 802, one or more millimeter wave and/or terahertz output signals are transmitted toward a surface. The one or more millimeter wave and/or terahertz output signals comprise millimeter wave radar output signals and frequencies in a range of from about 1 Hz to about 3,000,000 GHz. The one or more output signals transmitted toward a localized area of the surface suspected of containing virus particles and/or bacteria particles. The one or more output signals are configured to be absorbed by an ionic electrically charged particle with an electro-chemical valence shell on the surface. The ionic electrically charged particle with electro-chemical valence shell comprises a virus particle or a bacteria particle, for example. In some embodiments, operation 802 is performed by or with a processor and/or a transmitter similar to and/or the same as processor 110 and/or transmitter 106 (shown in FIG. 1 and described herein).

At an operation 804, a location of the particle is determined based on a frequency dependent electromagnetic response of the output signals reflected off of the surface. The determined location is a three-dimensional (3D) location of the particle. The frequency dependent electromagnetic response comprises a relative lack of energy at one or more frequencies of the output signals absorbed by the particle. The electromagnetic response is processed to determine an absorbed frequency indicated by the electromagnetic response, and determine a type of particle based on the absorbed frequency. In some embodiments, the location of the particle is determined using differential absorption spectroscopy. In some embodiments, the type of the particle comprises a type of virus, a type of bacteria, or a type of individual ionic particle. In some embodiments, operation 804 is performed by or with a processor and/or a receiver similar to and/or the same as processor 110 and/or receiver 108 (shown in FIG. 1 and described herein). In some embodiments, operation 704 is performed by or with a processor similar to and/or the same as processor 110 (shown in FIG. 1 and described herein).

At an operation 806, a visual representation of the particle is generated based on the determined location and/or other information. The visual representation may be displayed on the surface. Generating the visual representation of the particle and displaying it on the surface comprises rendering a point cloud (based on the determined location) in three-dimensional space with the processor and displaying the rendered point cloud. Displaying the rendered point cloud may comprise projecting the rendered point cloud onto the surface with an augmented reality projector as one example. The rendered point cloud may be displayed on the surface using augmented reality headsets, glasses or goggles as another example. The visual representation of the particle that is generated on the surface is performed in real-time or near real-time while a user views the surface. In some embodiments, operation 706 is performed by augmented reality glasses or goggles and/or a computing device the same as or similar to augmented reality goggles 602 and/or computing device 300 (shown in FIGS. 3 and 6A respectively and described herein).

The processor(s) described herein (e.g., 110, 310a . . .310n, etc.) may be configured to perform one or more of the operations described herein using a trained machine learning and/or other model. In some embodiments, the processor(s) are configured to cause a model to be trained using training information. In some embodiments, a model is trained by providing the training information as input to the model. In some embodiments, the model may be and/or include mathematical equations, algorithms, plots, charts, networks (e.g., neural networks), large language models, and/or other tools or components. For example, a machine learning model may be and/or include one or more neural networks having an input layer, an output layer, and one or more intermediate or hidden layers. In some embodiments, the one or more neural networks may be and/or include deep neural networks (e.g., neural networks that have one or more intermediate or hidden layers between the input and output layers).

As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function that combines the values of all its inputs together. In some embodiments, each connection (or the neural unit itself) may have a threshold function such that a signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free flowing, with connections interacting in a more chaotic and complex fashion.

As described above, the trained neural network may comprise one or more intermediate or hidden layers. The intermediate layers of the trained neural network include one or more convolutional layers, one or more recurrent layers, and/or other layers of the trained neural network. Individual intermediate layers receive information from another layer as input and generate corresponding outputs. In some embodiments, the trained neural network may comprise a deep neural network comprising a stack of convolution neural networks, followed by a stack of long short term memory (LSTM) elements, for example. The convolutional neural network layers may be thought of as filters, and the LSTM layers may be thought of as memory elements that keep track of data history, for example. The deep neural network may be configured such that there are max pooling layers which reduce dimensionality between the convolutional neural network layers. In some embodiments, the deep neural network comprises optional scalar parameters before the LSTM layers. In some embodiments, the deep neural network comprises dense layers, on top of the convolutional layers and recurrent layers. In some embodiments, the deep neural network may comprise additional hyper-parameters, such as dropouts or weight-regularization parameters, for example.

The one or more processors may similarly utilize large language models or other models commonly referred to as “AI models”.

Various embodiments of the present systems and methods are disclosed in the subsequent list of numbered clauses. In the following, further features, characteristics, and exemplary technical solutions of the present disclosure will be described in terms of clauses that may be optionally claimed in any combination.
1. A method for visualizing presence of virus and/or bacteria particles on a surface, the method comprising: transmitting one or more millimeter wave and/or terahertz output signals toward a surface, the one or more output signals configured to trigger a resonant frequency of an ionic electrically charged particle with an electro-chemical valence shell on the surface, wherein the ionic electrically charged particle with electro-chemical valence shell comprises a virus particle or a bacteria particle; determining a location of the particle based on a triggered frequency dependent electromagnetic response received from the particle; and generating a visual representation of the particle on the surface based on the location.
2. The method of clause 1, further comprising determining locations and generating visual representations of a plurality of virus and/or bacteria particles on the surface based on triggered frequency dependent electromagnetic responses received from the plurality of virus and/or bacteria particles.
3. The method of any of the previous clauses, further comprising transmitting the one or more output signals toward a localized area of the surface suspected of containing the virus particles and/or the bacteria particles.
4. The method of any of the previous clauses, wherein the electromagnetic response is received from virus particles and/or the bacteria particles, but not other particles, such that responsive to no virus particles and/or bacteria particles being present in the localized area, no electromagnetic response is received.
5. The method of any of the previous clauses, wherein the one or more millimeter wave and/or terahertz output signals comprise millimeter wave radar output signals.
6. The method of any of the previous clauses, wherein the one or more millimeter wave and/or terahertz output signals comprise frequencies in a range of from about 1 Hz to about 3,000,000 GHz.
7. The method of any of the previous clauses, further comprising processing the electromagnetic response to determine a peak frequency of the electromagnetic response from the particle, and determining a type of the particle based on the peak frequency.
8. The method of any of the previous clauses, wherein the type of the particle comprises a type of virus, a type of bacteria, or a type of individual ionic particle.
9. The method of any of the previous clauses, wherein the location is a three-dimensional (3D) location of the particle.
10. The method of any of the previous clauses, wherein the transmitting is performed by a nanometer scale complementary metal-oxide-semiconductor (CMOS) processor generating control signals to cause a phased array of antenna elements to generate the one or more output signals.
11. The method of any of the previous clauses, wherein the processor is configured to direct a digitally synthesized waveform through a digital to analog converter, and into the phased array of antenna elements for transmission, and wherein each of the phased array of antenna elements includes a phase shifter, a heterodyne series of up-converters where a high frequency local oscillator running at sub terahertz frequencies goes into a silicon power amplifier and into a silicon antenna element.
12. The method of any of the previous clauses, wherein the phased array is small enough to be included on an augmented reality device or worn on clothing.
13. The method of any of the previous clauses, wherein the processor tunes the local oscillator, and/or switches in multiple local oscillators to sweep a known set of frequencies at which ionic electrically charged particles with electro-chemical valence shells resonate.
14. The method of any of the previous clauses, wherein determining the location of the particle comprises: receiving the electromagnetic response from the particle with a millimeter wave and/or terahertz receiver, processing the electromagnetic response with the processor to determine whether a strength of the electromagnetic response breaches a threshold, and responsive to a threshold breach: using a radar ranging technique to determine a range of the particle from the phased array, tuning the phase shifters to generate a beam usable to map dimensional coordinates on the surface from where the electromagnetic response was received, and causing sweeping of beam and range gates in three dimensional space to map a point cloud of electromagnetic responses.
15. The method of any of the previous clauses, further comprising tagging, with the processor, the point cloud with a peak frequency associated with the point cloud, and correlating the peak frequency to a likely virus or bacteria type for the particle.
16. The method of any of the previous clauses, wherein the radar ranging technique comprises phase shift keying and range gates and pulse doppler techniques.
17. The method of any of the previous clauses, wherein generating the visual representation of the particle on the surface based on the location comprises rendering the point cloud in three dimensional space with the processor, and displaying the rendered point cloud.
18. The method of any of the previous clauses, wherein displaying the rendered point cloud comprises projecting the rendered point cloud onto the surface with an augmented reality projector.
19. The method of any of the previous clauses, wherein the rendered point cloud is displayed on the surface using augmented reality headsets or glasses or goggles.
20. The method of any of the previous clauses, wherein generating the visual representation of the particle on the surface is performed in real-time or near real-time while a user views the surface.
21. A system for visualizing presence of virus and/or bacteria particles on a surface, the system comprising: a transmitter configured to transmit one or more millimeter wave and/or terahertz output signals toward a surface, the one or more output signals configured to trigger a resonant frequency of an ionic electrically charged particle with an electro-chemical valence shell on the surface, wherein the ionic electrically charged particle with electro-chemical valence shell comprises a virus particle or a bacteria particle; a receiver configured to receive a triggered frequency dependent electromagnetic response received from the particle; and a processor configured to: determine a location of the particle based on the electromagnetic response; and generate a visual representation of the particle on the surface based on the location.
22. The system of clause 21, wherein the processor is further configured to determine locations and generate visual representations of a plurality of virus and/or bacteria particles on the surface based on triggered frequency dependent electromagnetic responses received from the plurality of virus and/or bacteria particles.
23. The system of any of the previous clauses, wherein the transmitter is configured to transmit the one or more output signals toward a localized area of the surface suspected of containing the virus particles and/or the bacteria particles.
24. The system of any of the previous clauses, wherein the electromagnetic response is received from virus particles and/or the bacteria particles, but not other particles, such that responsive to no virus particles and/or bacteria particles being present in the localized area, no electromagnetic response is received.
25. The system of any of the previous clauses, wherein the one or more millimeter wave and/or terahertz output signals comprise millimeter wave radar output signals.
26. The system of any of the previous clauses, wherein the one or more millimeter wave and/or terahertz output signals comprise frequencies in a range of from about 1 Hz to about 3,000,000 GHz.
27. The system of any of the previous clauses, wherein the processor is further configured to process the electromagnetic response to determine a peak frequency of the electromagnetic response from the particle, and determine a type of the particle based on the peak frequency.
28. The system of any of the previous clauses, wherein the type of the particle comprises a type of virus, a type of bacteria, or a type of individual ionic particle.
29. The system of any of the previous clauses, wherein the location is a three-dimensional (3D) location of the particle.
30. The system of any of the previous clauses, wherein processor is a nanometer scale complementary metal-oxide-semiconductor (CMOS) processor configured to generate control signals to cause a phased array of antenna elements in the transmitter to generate the one or more output signals.
31. The system of any of the previous clauses, wherein the processor is configured to direct a digitally synthesized waveform through a digital to analog converter, and into the phased array of antenna elements for transmission, and wherein each of the phased array of antenna elements includes a phase shifter, a heterodyne series of up-converters where a high frequency local oscillator running at sub terahertz frequencies goes into a silicon power amplifier and into a silicon antenna element.
32. The system of any of the previous clauses, wherein the phased array is small enough to be included on an augmented reality device or worn on clothing.
33. The system of any of the previous clauses, wherein the processor tunes the local oscillator, and/or switches in multiple local oscillators to sweep a known set of frequencies at which ionic electrically charged particles with electro-chemical valence shells resonate.
34. The system of any of the previous clauses, wherein the receiver is a millimeter wave and/or terahertz receiver, and the processor is configured to process the electromagnetic response to determine whether a strength of the electromagnetic response breaches a threshold, and responsive to a threshold breach: use a radar ranging technique to determine a range of the particle from the phased array, tune the phase shifters to generate a beam usable to map dimensional coordinates on the surface from where the electromagnetic response was received, and cause sweeping of beam and range gates in three dimensional space to map a point cloud of electromagnetic responses.
35. The system of any of the previous clauses, wherein the processor is further configured to tag the point cloud with a peak frequency associated with the point cloud, and correlate the peak frequency to a likely virus or bacteria type for the particle.
36. The system of any of the previous clauses, wherein the radar ranging technique comprises phase shift keying and range gates and pulse doppler techniques.
37. The system of any of the previous clauses, wherein generating the visual representation of the particle on the surface based on the location comprises rendering the point cloud in three dimensional space with the processor, and displaying the rendered point cloud.
38. The system of any of the previous clauses, further comprising an augmented reality projector configured to project the rendered point cloud onto the surface.
39. The system of any of the previous clauses, further comprising augmented reality headsets or glasses or goggles configured to display the rendered point on the surface.
40. The system of any of the previous clauses, wherein generating the visual representation of the particle on the surface is performed in real-time or near real-time while a user of the system views the surface.
41. A method for visualizing presence of virus and/or bacteria particles on a surface, the method comprising: transmitting one or more millimeter wave and/or terahertz output signals toward a surface, the one or more output signals configured to be absorbed by an ionic electrically charged particle with an electro-chemical valence shell on the surface, wherein the ionic electrically charged particle with electro-chemical valence shell comprises a virus particle or a bacteria particle; determining a location of the particle based on a frequency dependent electromagnetic response of the output signals reflected off of the surface, wherein the frequency dependent electromagnetic response comprises a relative lack of energy at one or more frequencies of the output signals absorbed by the particle; and generating a visual representation of the particle on the surface based on the location.
42. The method of any of the previous clauses, further comprising determining locations and generating visual representations of a plurality of virus and/or bacteria particles on the surface based on absorbed frequency dependent electromagnetic responses received from the plurality of virus and/or bacteria particles.
43. The method of any of the previous clauses, further comprising transmitting the one or more output signals toward a localized area of the surface suspected of containing the virus particles and/or the bacteria particles.
44. The method of any of the previous clauses, wherein the location of the particle is determined using differential absorption spectroscopy.
45. The method of any of the previous clauses, wherein the one or more millimeter wave and/or terahertz output signals comprise millimeter wave radar output signals.
46. The method of any of the previous clauses, wherein the one or more millimeter wave and/or terahertz output signals comprise frequencies in a range of from about 1 Hz to about 3,000,000 GHz.
47. The method of any of the previous clauses, further comprising processing the electromagnetic response to determine an absorbed frequency indicated by the electromagnetic response, and determining a type of the particle based on the absorbed frequency.
48. The method of any of the previous clauses, wherein the type of the particle comprises a type of virus, a type of bacteria, or a type of individual ionic particle.
49. The method of any of the previous clauses, wherein the location is a three-dimensional (3D) location of the particle, and generating the visual representation of the particle on the surface comprises: projecting a rendered point cloud that represents the particle onto the surface with an augmented reality projector; or displaying an image of the particle on the surface using augmented reality headsets or glasses or goggles.
50. The method of any of the previous clauses, wherein generating the visual representation of the particle on the surface is performed in real-time or near real-time while a user views the surface.
51. A system for visualizing presence of virus and/or bacteria particles on a surface, the system comprising: a transmitter configured to transmit one or more millimeter wave and/or terahertz output signals toward a surface, the one or more output signals configured to be absorbed by an ionic electrically charged particle with an electro-chemical valence shell on the surface, wherein the ionic electrically charged particle with electro-chemical valence shell comprises a virus particle or a bacteria particle; a receiver configured to receive a frequency dependent electromagnetic response received from the surface, wherein the frequency dependent electromagnetic response comprises a relative lack of energy at one or more frequencies of the output signals absorbed by the particle; and a processor configured to: determine a location of the particle based on the frequency dependent electromagnetic response; and generate a visual representation of the particle on the surface based on the location.
52. The system of any of the previous clauses, wherein the processor is further configured to determine locations and generate visual representations of a plurality of virus and/or bacteria particles on the surface based on absorbed frequency dependent electromagnetic responses received from the plurality of virus and/or bacteria particles.
53. The system of any of the previous clauses, wherein the transmitter is configured to transmit the one or more output signals toward a localized area of the surface suspected of containing the virus particles and/or the bacteria particles.
54. The system of any of the previous clauses, wherein the location of the particle is determined using differential absorption spectroscopy by the processor.
55. The system of any of the previous clauses, wherein the one or more millimeter wave and/or terahertz output signals comprise millimeter wave radar output signals.
56. The system of any of the previous clauses, wherein the one or more millimeter wave and/or terahertz output signals comprise frequencies in a range of from about 1 Hz to about 3,000,000 GHz.
57. The system of any of the previous clauses, wherein the processor is further configured to process the electromagnetic response to determine an absorbed frequency indicated by the electromagnetic response, and determine a type of the particle based on the absorbed frequency.
58. The system of any of the previous clauses, wherein the type of the particle comprises a type of virus, a type of bacteria, or a type of individual ionic particle.
59. The system of any of the previous clauses, wherein the location is a three-dimensional (3D) location of the particle, and generating the visual representation of the particle on the surface comprises: projecting a rendered point cloud that represents the particle onto the surface with an augmented reality projector controlled by the processor; or displaying an image of the particle on the surface using augmented reality headsets or glasses or goggles controlled by the processor.
60. The system of any of the previous clauses, wherein generating the visual representation of the particle on the surface is performed in real-time or near real-time by the processor while a user views the surface.

The reader should appreciate that the present application describes several inventions. Rather than separating those inventions into multiple isolated patent applications, applicants have grouped these inventions into a single document because their related subject matter lends itself to economies in the application process. But the distinct advantages and aspects of such inventions should not be conflated. In some cases, embodiments address all of the deficiencies noted herein, but it should be understood that the inventions are independently useful, and some embodiments address only a subset of such problems or offer other, unmentioned benefits that will be apparent to those of skill in the art reviewing the present disclosure. Due to cost constraints, some inventions disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such inventions or all aspects of such inventions.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more. ” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device.

What is claimed is:

1. A method for visualizing presence of virus and/or bacteria particles on a surface, the method comprising:
transmitting one or more millimeter wave and/or terahertz output signals toward a surface, the one or more output signals configured to trigger a resonant frequency of an ionic electrically charged particle with an electro-chemical valence shell on the surface, wherein the ionic electrically charged particle with electro-chemical valence shell comprises a virus particle or a bacteria particle;
determining a location of the particle based on a triggered frequency dependent electromagnetic response received from the particle; and
generating a visual representation of the particle on the surface based on the location.

2. The method of claim 1, further comprising determining locations and generating visual representations of a plurality of virus and/or bacteria particles on the surface based on triggered frequency dependent electromagnetic responses received from the plurality of virus and/or bacteria particles.

3. The method of claim 2, further comprising transmitting the one or more output signals toward a localized area of the surface suspected of containing the virus particles and/or the bacteria particles.

4. The method of claim 3, wherein the electromagnetic response is received from virus particles and/or the bacteria particles, but not other particles, such that responsive to no virus particles and/or bacteria particles being present in the localized area, no electromagnetic response is received.

5. The method of claim 1, wherein the one or more millimeter wave and/or terahertz output signals comprise millimeter wave radar output signals.

6. The method of claim 1, wherein the one or more millimeter wave and/or terahertz output signals comprise frequencies in a range of from about 1 Hz to about 3,000,000 GHz.

7. The method of claim 1, further comprising processing the electromagnetic response to determine a peak frequency of the electromagnetic response from the particle and determining a type of the particle based on the peak frequency.

8. The method of claim 7, wherein the type of the particle comprises a type of virus, a type of bacteria, or a type of individual ionic particle.

9. The method of claim 1, wherein the location is a three-dimensional (3D) location of the particle.

10. The method of claim 1, wherein the transmitting is performed by a nanometer scale complementary metal-oxide-semiconductor (CMOS) processor generating control signals to cause a phased array of antenna elements to generate the one or more output signals.

11. The method of claim 10, wherein the processor is configured to direct a digitally synthesized waveform through a digital to analog converter, and into the phased array of antenna elements for transmission, and wherein each of the phased array of antenna elements includes a phase shifter, a heterodyne series of up-converters where a high frequency local oscillator running at sub terahertz frequencies goes into a silicon power amplifier and into a silicon antenna element.

12. The method of claim 11, wherein the phased array is small enough to be included on an augmented reality device or worn on clothing.

13. The method of claim 11, wherein the processor tunes the local oscillator, and/or switches in multiple local oscillators to sweep a known set of frequencies at which ionic electrically charged particles with electro-chemical valence shells resonate.

14. The method of claim 11, wherein determining the location of the particle comprises:
receiving the electromagnetic response from the particle with a millimeter wave and/or terahertz receiver,
processing the electromagnetic response with the processor to determine whether a strength of the electromagnetic response breaches a threshold, and responsive to a threshold breach:
using a radar ranging technique to determine a range of the particle from the phased array,
tuning the phase shifters to generate a beam usable to map dimensional coordinates on the surface from where the electromagnetic response was received, and
causing sweeping of beam and range gates in three dimensional space to map a point cloud of electromagnetic responses.

15. The method of claim 14, further comprising tagging, with the processor, the point cloud with a peak frequency associated with the point cloud, and correlating the peak frequency to a likely virus or bacteria type for the particle.

16. The method of claim 14, wherein the radar ranging technique comprises phase shift keying and range gates and pulse doppler techniques.

17. The method of claim 14, wherein generating the visual representation of the particle on the surface based on the location comprises rendering the point cloud in three dimensional space with the processor, and displaying the rendered point cloud.

18. The method of claim 17, wherein displaying the rendered point cloud comprises projecting the rendered point cloud onto the surface with an augmented reality projector.

19. The method of claim 17, wherein the rendered point cloud is displayed on the surface using augmented reality headsets or glasses or goggles.

20. The method of claim 1, wherein generating the visual representation of the particle on the surface is performed in real-time or near real-time while a user views the surface.

21. A system for visualizing presence of virus and/or bacteria particles on a surface, the system comprising:
a transmitter configured to transmit one or more millimeter wave and/or terahertz output signals toward a surface, the one or more output signals configured to trigger a resonant frequency of an ionic electrically charged particle with an electro-chemical valence shell on the surface, wherein the ionic electrically charged particle with electro-chemical valence shell comprises a virus particle or a bacteria particle;
a receiver configured to receive a triggered frequency dependent electromagnetic response received from the particle; and
a processor configured to:
determine a location of the particle based on the electromagnetic response; and
generate a visual representation of the particle on the surface based on the location.

22. The system of claim 21, wherein the processor is further configured to determine locations and generate visual representations of a plurality of virus and/or bacteria particles on the surface based on triggered frequency dependent electromagnetic responses received from the plurality of virus and/or bacteria particles.

23. The system of claim 22, wherein the transmitter is configured to transmit the one or more output signals toward a localized area of the surface suspected of containing the virus particles and/or the bacteria particles.

24. The system of claim 23, wherein the electromagnetic response is received from virus particles and/or the bacteria particles, but not other particles, such that responsive to no virus particles and/or bacteria particles being present in the localized area, no electromagnetic response is received.

25. The system of claim 21, wherein the one or more millimeter wave and/or terahertz output signals comprise millimeter wave radar output signals.

26. The system of claim 21, wherein the one or more millimeter wave and/or terahertz output signals comprise frequencies in a range of from about 1 Hz to about 3,000,000 GHz.

27. The system of claim 21, wherein the processor is further configured to process the electromagnetic response to determine a peak frequency of the electromagnetic response from the particle, and determine a type of the particle based on the peak frequency.

28. The system of claim 27, wherein the type of the particle comprises a type of virus, a type of bacteria, or a type of individual ionic particle.

29. The system of claim 21, wherein the location is a three-dimensional (3D) location of the particle.

30. The system of claim 21, wherein processor is a nanometer scale complementary metal-oxide-semiconductor (CMOS) processor configured to generate control signals to cause a phased array of antenna elements in the transmitter to generate the one or more output signals.

31. The system of claim 30, wherein the processor is configured to direct a digitally synthesized waveform through a digital to analog converter, and into the phased array of antenna elements for transmission, and wherein each of the phased array of antenna elements includes a phase shifter, a heterodyne series of up-converters where a high frequency local oscillator running at sub terahertz frequencies goes into a silicon power amplifier and into a silicon antenna element.

32. The system of claim 31, wherein the phased array is small enough to be included on an augmented reality device or worn on clothing.

33. The system of claim 31, wherein the processor tunes the local oscillator, and/or switches in multiple local oscillators to sweep a known set of frequencies at which ionic electrically charged particles with electro-chemical valence shells resonate.

34. The system of claim 31, wherein the receiver is a millimeter wave and/or terahertz receiver, and the processor is configured to process the electromagnetic response to determine whether a strength of the electromagnetic response breaches a threshold, and responsive to a threshold breach:
use a radar ranging technique to determine a range of the particle from the phased array,
tune the phase shifters to generate a beam usable to map dimensional coordinates on the surface from where the electromagnetic response was received, and
cause sweeping of beam and range gates in three dimensional space to map a point cloud of electromagnetic responses.

35. The system of claim 34, wherein the processor is further configured to tag the point cloud with a peak frequency associated with the point cloud, and correlate the peak frequency to a likely virus or bacteria type for the particle.

36. The system of claim 34, wherein the radar ranging technique comprises phase shift keying and range gates and pulse doppler techniques.

37. The system of claim 34, wherein generating the visual representation of the particle on the surface based on the location comprises rendering the point cloud in three dimensional space with the processor, and displaying the rendered point cloud.

38. The system of claim 37, further comprising an augmented reality projector configured to project the rendered point cloud onto the surface.

39. The system of claim 37, further comprising augmented reality headsets or glasses or goggles configured to display the rendered point on the surface.

40. The system of claim 21, wherein generating the visual representation of the particle on the surface is performed in real-time or near real-time while a user of the system views the surface.

41. A method for visualizing presence of virus and/or bacteria particles on a surface, the method comprising:
transmitting one or more millimeter wave and/or terahertz output signals toward a surface, the one or more output signals configured to be absorbed by an ionic electrically charged particle with an electro-chemical valence shell on the surface, wherein the ionic electrically charged particle with electro-chemical valence shell comprises a virus particle or a bacteria particle;
determining a location of the particle based on a frequency dependent electromagnetic response of the output signals reflected off of the surface, wherein the frequency dependent electromagnetic response comprises a relative lack of energy at one or more frequencies of the output signals absorbed by the particle; and
generating a visual representation of the particle on the surface based on the location.

42. The method of claim 41, further comprising determining locations and generating visual representations of a plurality of virus and/or bacteria particles on the surface based on absorbed frequency dependent electromagnetic responses received from the plurality of virus and/or bacteria particles.

43. The method of claim 42, further comprising transmitting the one or more output signals toward a localized area of the surface suspected of containing the virus particles and/or the bacteria particles.

44. The method of claim 43, wherein the location of the particle is determined using differential absorption spectroscopy.

45. The method of claim 41, wherein the one or more millimeter wave and/or terahertz output signals comprise millimeter wave radar output signals.

46. The method of claim 41, wherein the one or more millimeter wave and/or terahertz output signals comprise frequencies in a range of from about 1 Hz to about 3,000,000 GHz.

47. The method of claim 41, further comprising processing the electromagnetic response to determine an absorbed frequency indicated by the electromagnetic response, and determining a type of the particle based on the absorbed frequency.

48. The method of claim 47, wherein the type of the particle comprises a type of virus, a type of bacteria, or a type of individual ionic particle.

49. The method of claim 41, wherein the location is a three-dimensional (3D) location of the particle, and generating the visual representation of the particle on the surface comprises:
projecting a rendered point cloud that represents the particle onto the surface with an augmented reality projector; or
displaying an image of the particle on the surface using augmented reality headsets or glasses or goggles.

50. The method of claim 41, wherein generating the visual representation of the particle on the surface is performed in real-time or near real-time while a user views the surface.

51. A system for visualizing presence of virus and/or bacteria particles on a surface, the system comprising:
a transmitter configured to transmit one or more millimeter wave and/or terahertz output signals toward a surface, the one or more output signals configured to be absorbed by an ionic electrically charged particle with an electro-chemical valence shell on the surface, wherein the ionic electrically charged particle with electro-chemical valence shell comprises a virus particle or a bacteria particle;
a receiver configured to receive a frequency dependent electromagnetic response received from the surface, wherein the frequency dependent electromagnetic response comprises a relative lack of energy at one or more frequencies of the output signals absorbed by the particle; and
a processor configured to:
determine a location of the particle based on the frequency dependent electromagnetic response; and
generate a visual representation of the particle on the surface based on the location.

52. The system of claim 51, wherein the processor is further configured to determine locations and generate visual representations of a plurality of virus and/or bacteria particles on the surface based on absorbed frequency dependent electromagnetic responses received from the plurality of virus and/or bacteria particles.

53. The system of claim 52, wherein the transmitter is configured to transmit the one or more output signals toward a localized area of the surface suspected of containing the virus particles and/or the bacteria particles.

54. The system of claim 53, wherein the location of the particle is determined using differential absorption spectroscopy by the processor.

55. The system of claim 51, wherein the one or more millimeter wave and/or terahertz output signals comprise millimeter wave radar output signals.

56. The system of claim 51, wherein the one or more millimeter wave and/or terahertz output signals comprise frequencies in a range of from about 1 Hz to about 3,000,000 GHz.

57. The system of claim 51, wherein the processor is further configured to process the electromagnetic response to determine an absorbed frequency indicated by the electromagnetic response, and determine a type of the particle based on the absorbed frequency.

58. The system of claim 57, wherein the type of the particle comprises a type of virus, a type of bacteria, or a type of individual ionic particle.

59. The system of claim 51, wherein the location is a three-dimensional (3D) location of the particle, and generating the visual representation of the particle on the surface comprises:
projecting a rendered point cloud that represents the particle onto the surface with an augmented reality projector controlled by the processor; or
displaying an image of the particle on the surface using augmented reality headsets or glasses or goggles controlled by the processor.

60. The system of claim 51, wherein generating the visual representation of the particle on the surface is performed in real-time or near real-time by the processor while a user views the surface.
